(19) United States
(12) Patent Application Publication
(10) Pub. No.: US 2023/0404427 A1
(43) Pub. Date: Dec. 21, 2023

(54) Techniques For Tracking Ingestible Device

(71) Applicant: THE REGENTS OF THE UNIVERSITY OF MICHIGAN, Ann Arbor, MI (US)

(72) Inventors: Duxin SUN, Ann Arbor, MI (US); Yogesh GIANCHANDANI, Ann Arbor, MI (US); Tao LI, Ann Arbor, MI (US); Jeremy FELTON, Ann Arbor, MI (US); Hsueh-Tsung LU, Ann Arbor, MI (US); Alexander BENKEN, Ann Arbor, MI (US); Partha DUTTA, Ann Arbor, MI (US); Xiangyu ZHAO, Ann Arbor, MI (US); Manjunath PAI, Ann Arbor, MI (US)

(73) Assignee: THE REGENTS OF THE UNIVERSITY OF MICHIGAN, Ann Arbor, MI (US)

(21) Appl. No.: 18/210,980

(22) Filed: Jun. 16, 2023

Related U.S. Application Data
(60) Provisional application No. 63/353,056, filed on Jun. 17, 2022.

(51) Int. Cl. A61B 5/06; A61B 5/00; A61B 5/07

(52) U.S. Cl. CPC A61B 5/067; A61B 5/6861; A61B 5/4839; A61B 5/073; A61B 2010/0061

(57) ABSTRACT

A method is presented for tracking an ingestible device in a gastrointestinal tract of a subject. The method includes: measuring acceleration of the ingestible device as it traverses through the gastrointestinal tract, for example using an accelerometer disposed in the ingestible device; computing a metric from the acceleration measurements obtained by the accelerometer over a given period of time; and identifying a location in the gastrointestinal tract based in part on the metric.

### GOVERNMENT CLAUSE

This invention was made with government support under HHSF223201510146C awarded by the U.S. Food and Drug Administration. The government has certain rights in the invention.

### FIELD

The present disclosure relates to techniques for tracking an ingestible device in a gastrointestinal track of a subject.

### BACKGROUND

Sampling and analysis of fluids from multiple locations along the gastrointestinal (GI) tract, for example stomach, small intestine, and colon, are of significant interest in oral drug product development and in the understanding and diagnosis of GI conditions and diseases. For oral drug product development, it is desired to estimate drug release in the different regions of GI tract by obtaining and analyzing GI samples. The in vivo data on drug release in the GI tract will help understand drug product quality, oral drug absorption and pharmacokinetics to ensure drug efficacy and safety. For diagnosis of GI conditions and disease, it is ideal to analyze different biomarkers (such as bile salts) from samples of different regions of GI tract. These biomarker changes in the GI tract will reflect GI disease conditions such as cancer, bacterial infections (e.g. H pylori and C. difficile), ulcer, GI bleeding, bacteria overgrowth, etc. However, it is not only very challenging to obtain GI samples from different regions of GI tract, but also difficult to know the locations where the samples are collected. The traditional method to obtain GI samples for these analyses is through GI intubation using specially designed catheters. These catheters have specially designed openings to collect fluids from multiple sites along the GI tract but have only limited reach beyond the stomach into the small intestine. Additionally, the procedure is challenging by nature, can cause discomfort, requires medical supervision and anesthesia, and therefore is not suitable for wide routine use.

An ingestible sampling device that can collect and store multiple fluid samples along the GI tract is highly desirable to meet this gap, providing a solution that is noninvasive and applicable to the whole GI tract. The device should collect fluid samples from multiple identified sites along the GI tract to measure concentration of drugs and biomarkers (such as bile salts) with both spatial and temporal information. Ingestible devices for sensing or imaging in the GI tract have a long history with a number of devices developed in either commercial settings (e.g. SmartPill® and PillCam®) or research frontiers. The SmartPill, which has both motility sensor and pH sensor inside the capsule, is used in clinical patients to record the motility and pH data. The PillCam, which has a small camera inside a capsule, is used in human patients to take images of GI tract as endoscopy. However, neither SmartPill nor PillCam is able to take samples from GI tract. In addition, these two devices are unable to track their location in the GI tract.

Another ingestible device, IntelliCap®, has been developed for drug delivery and sensing of pH and temperature in the GI tract. There are currently no commercially available ingestible devices for fluid sampling in the GI tract while a few research prototypes have been reported that at best partially meet the needs.

A capsule developed using microelectromechanical systems (MEMS) technologies can perform simultaneous drug delivery and fluid sampling by thermally actuating a piston between two reservoirs, one for the drug and the other for the collected fluid. Two passive devices were reported for collection of microbiome samples from the small intestine, both using an enteric coating that blocks sampling inlets until the coating dissolves at the basic pH levels in the small intestine; one device used hydrogel to absorb GI fluid with microbiome samples, whereas the other trapped microorganism samples in channels from which water was drained osmotically. Another approach is to incorporate a motor within the capsule to selectively rotate a sampling port across multiple storage chambers.

In addition to the challenge of sampling fluid from different locations in the GI tract, a tracking mechanism is necessary to maintain spatial information of collected fluid samples. There can be wide variations in gastric emptying time (GET) among individuals and in different body conditions. Various tracking and imaging techniques have been reported for ingestible devices, ranging from techniques that monitor variations in magnetic field, RF waves or visible light, to x-ray or magnetic resonance imaging. The most widely used technique is magnetic field tracking, where typically the magnetic field generated by a small permanent magnet embedded in the device is tracked by an array of magnetic sensors, such as Hall sensors, arranged outside the body near the waist. The recorded variations in the magnetic field at each sensor depend on the location and orientation of the sensor relative to the device and can be used to derive the three dimensional (3D) position of the device by using complicated algorithm, e.g. a solution available from Motilis SA, Switzerland to process large amount of sensor data.

In this disclosure, miniature inertial sensors are used to monitor instantaneous acceleration patterns of the device as it travels along the GI tract. By comparing the recorded acceleration patterns with the distinct motility patterns in stomach, small intestine, and colon, respectively, this technique can identify the GI tract segment in which the device is located, meeting the needs of location tracking for the ingestible sampling device without having to use external imaging hardware and physically confine the subject.

This section provides background information related to the present disclosure which is not necessarily prior art.

### SUMMARY

This section provides a general summary of the disclosure, and is not a comprehensive disclosure of its full scope or all of its features.

In one aspect, a method is presented for tracking an ingestible device in a gastrointestinal tract of a subject. The method includes: measuring, by an accelerometer disposed in the ingestible device, acceleration of the ingestible device as it traverses through the gastrointestinal tract; computing a metric from the acceleration measurements obtained by the accelerometer over a given period of time; and identifying a location in the gastrointestinal tract based in part on the metric.

In another aspect, an ingestible device is presented for traversing a gastrointestinal tract. The device includes: a housing configured to be ingested by a subject; an accelerometer disposed inside the housing and operable to measure acceleration of the ingestible device as it traverses through the gastrointestinal tract of the subject; and a microcontroller residing in the housing and interfaced with the accelerometer. The microcontroller is configured to computer a metric from the acceleration measurements obtained by the accelerometer over a period of time and identify a location in the gastrointestinal tract based on the metric.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a cross-sectional perspective view of an example ingestible electronic capsule according to certain aspects of the present disclosure;

FIG. 2 is an exploded perspective view of an example ingestible electronic capsule according to certain aspects of the present disclosure;

FIG. 3 is a cross-sectional perspective view of the example ingestible electronic capsule of FIG. 2;

FIG. 4 is a block diagram of the components comprising an ingestible device;

FIG. 5 is a state diagram for the operation of an ingestible device;

FIG. 6 is a flowchart depicting an example method for tracking an ingestible device in a gastrointestinal tract of a subject;

FIG. 7 is a diagram illustrating tracking and sampling events by an ingestible device during an in vivo test;

FIGS. 8 and 9 are graphs showing acceleration measurements during the in vivo test;

FIG. 10 shows in vivo test results for bile salt concentration taken in stomach of a hound;

FIG. 11 shows in vivo test results for bile salt concentration taken in small intestine of a hound; and

FIG. 12 shows in vivo test results for bile salt concentration taken in colon of a hound.

Corresponding reference numerals indicate corresponding parts throughout the several views of the drawings.

### DETAILED DESCRIPTION

Example embodiments will now be described more fully with reference to the accompanying drawings.

Among other features, the present disclosure provides an autonomous wireless sampling device in the form of an ingestible electronic capsule or pill for fluid collection within the gastric-intestinal tract allowing, inter alia, in vivo drug dissolution monitoring to aid the design of oral medications and treatments and generic drug products and location specific microbiota analysis. The sampling device may collect multiple specimens or samples of gastric-intestinal tract fluid and store these samples in isolated chambers or cartridges. After the ingestible electronic pill is expelled and cleaned, the collected samples may be extracted from the chambers for analysis. Further, wireless communication between the sampling device and an external unit may allow remote triggering of the sampling action as well as constant monitoring of the ingestible electronic device during deployment. In various instances, the sampling device may obtain specimens from one or more of the stomach, small intestine, large intestine, colon, and combinations thereof.

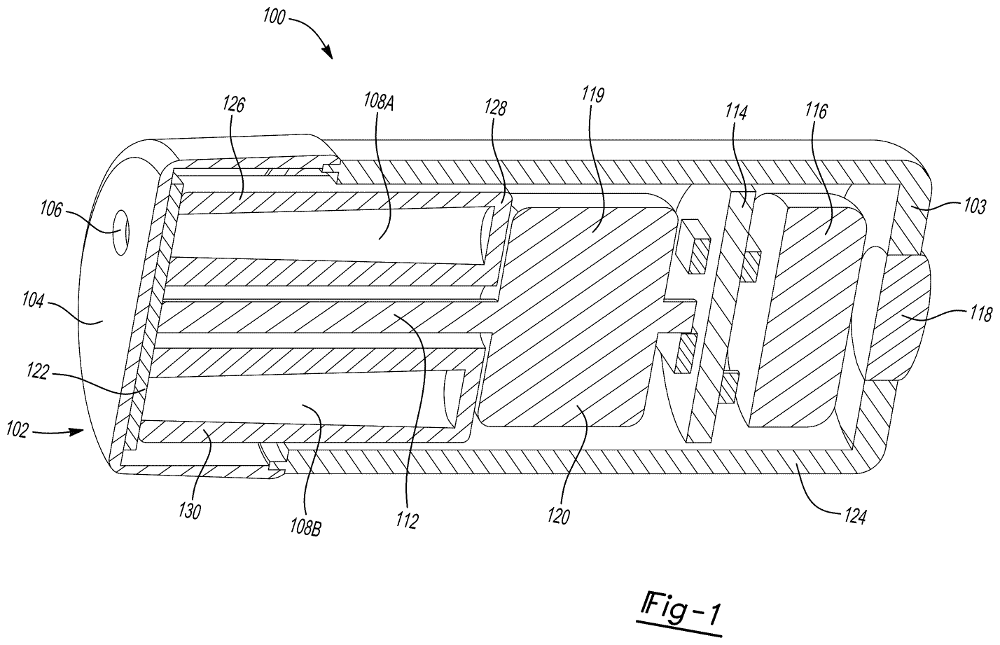

An exemplary and schematic illustration of an ingestible sampling device (or capsule) 100 is shown in FIG. 1. The sampling device 100 is designed to be ingested by the subject (i.e., human or animal) and passed through the subject's gastric-intestinal tract before being expelled as waste. For example, in certain embodiments, the sampling device 100 has a length of greater than or equal to about 25 mm to less than or equal to about 30 mm and a width or diameter of greater than or equal to about 10 mm to less than or equal to about 14 mm. In some embodiments, the diameter of the sampling device 100 may vary along the length of the sampling device 100. For example, a first end 104 of the sampling device 100 may have a first diameter that is less than or greater than a second diameter of a second end 103 of the sampling device 100. The (emptied) sampling device 100 may weigh greater than or equal to about 5 grams to less than or equal to about 15 grams.

The sampling device 100 includes a cap 102 and a housing element 124 connected thereto to form an interior chamber 119. The cap 102 includes a first end or surface 104 having a sampling port 106 formed therein. The first surface 104 of the cap 102 is parallel with a second end or surface 103 of the housing element 124. The second surface 103 of the housing element 124 includes an access port 118, which may be used to access components within the sampling device 100 for purposes of performing maintenance or the like (e.g., battery replacement).

One or more sample collection chambers (e.g., 108A and 108B) are disposed within the interior chamber 119 formed between the first end 104 and the second end 103. Although only two sample collection chambers 108 are shown in FIG. 1, according to some examples, eight or more sample collection chambers 108 may be included within the sampling device 100 without deviating from the teachings herein. Each sample collection chamber 108 includes a first end 126, a second end 128, and sidewalls 130 connecting the first and second ends 126, 128. The sample collection chambers 108 are configured to collect gastric-intestinal fluids from a subject's gastric-intestinal tract when the first end 126 of the sample collection chamber 108 is aligned with the sampling port 106 and exposed to the subject's gastric-intestinal tract. According to some examples, the one or more sample collection chambers 108 may collect one or more gastric-intestinal tract samples by virtue of a capillary force and/or suction force that draws the gastric-intestinal fluid samples into the sample collection chambers 108. In certain aspects, the one or more sample collection chamber 108 may each collect an amount of gastric-intestinal fluid ranging from about 5 μL to about 500 μL.

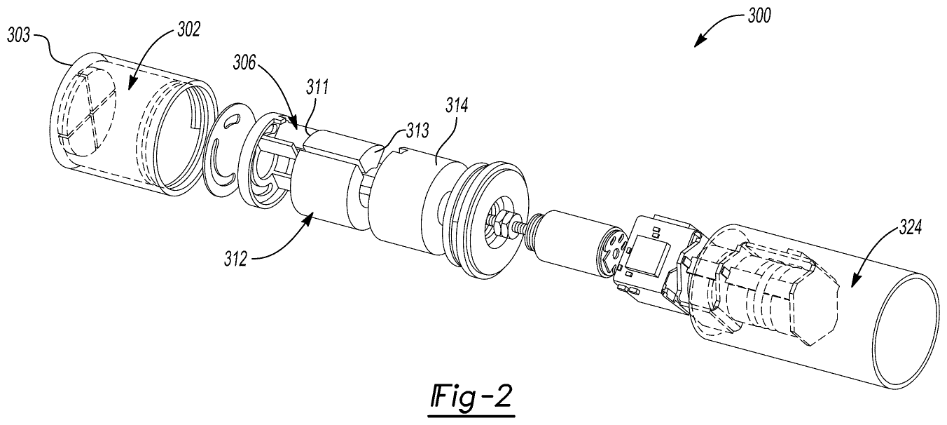

In certain aspects, the sample collection chambers 108 may be emptied prior to ingestion and sample collection. In other aspects (as seen for example in FIG. 2), each sample collection chamber 108 may include one or more foam-like cartridges 312 that absorb and retain the gastric-intestinal samples. In still other aspects, the sample collection chambers 108 may further include one or more sealing coating that coats one or more of the first end 126, the second end 128 and the sidewalls 130 of each sample collection chamber 108. For example, with specific reference to cartridge 108A, the second end 128 and sidewalls 130 may be coated with the sealing coating (not shown), while the first end 126 may remain coating-free to facilitate sample collection (e.g., when the sampling port 106 is aligned with the sample collection chamber 108a). The sealing coating may comprise an impervious polymer, such as rubber and may prevent cross contamination between samples within the adjacent sample collection chambers 108. In certain instances, a sealing layer 122 may also be disposed between the first ends 126 of the sample collection chambers 108 and the cap 102. The sealing layer 122 may be configured to prevent, for example, (i) unwanted ingress and egress of fluid (e.g., gastric-intestinal fluids) in and out of the sampling device 100 and (ii) cross contamination between the various sample collection chambers 108. The sealing layer 122 may be manufactured from any suitable material known in the art for accomplishing one or more of the foregoing objectives including, for example, plastic (e.g., polyimide) or grease (e.g., silicon grease).

With continued reference to FIG. 1, the sampling device 100 includes a rotatable shaft 112 that connects the cap 102 to a motor 120. The rotatable shaft 112 and motor 120 may be configured to rotate the cap 102 axially about the rotatable shaft 112 so that the sampling port 106 aligns with one of the sample collection chambers 108. In some embodiments, the sampling device 100 may exclude a rotatable shaft 112 and the motor 120 may be coupled to the one or more sample collection chambers 108. In such instances, the motor 120 may be configured to rotate the one or more sample collection chambers 108 relative to the sampling port 106. As such, according to some examples, the motor 120 may include one of a miniature stepper motor, a linear motor, or the like.

The sampling device 100 also includes a microcontroller 114 that is operatively connected (i.e., directly connected or connected via one or more intermediate components) to the motor 120. In some examples, the microcontroller 114 may be included on a circuit board (e.g., a PCB). The microcontroller 114 may include one or more processors (not shown) and memory (not shown) and is configured to control the motor 120. For example, according to some implementations, the microcontroller 114 may be configured to instruct the motor 120 to actuate so as to turn the rotatable shaft 112 and, consequently, the cap 102 by virtue of its connection to the rotatable shaft 112. In this manner, the microcontroller 114 may be configured to selectively align the sampling port 106 of the cap 102 with a given chamber (e.g., chamber 108a), thereby exposing the chamber (e.g., chamber 108a) to the gastric-intestinal tract for sample collection. The sample device 100 also include a battery 116 that is configured to supply power to the microcontroller 114 and/or motor 120. According to some examples, the battery 116 may provide an operational time of not less than about 40 hours of deployment within the gastric-intestinal tract.

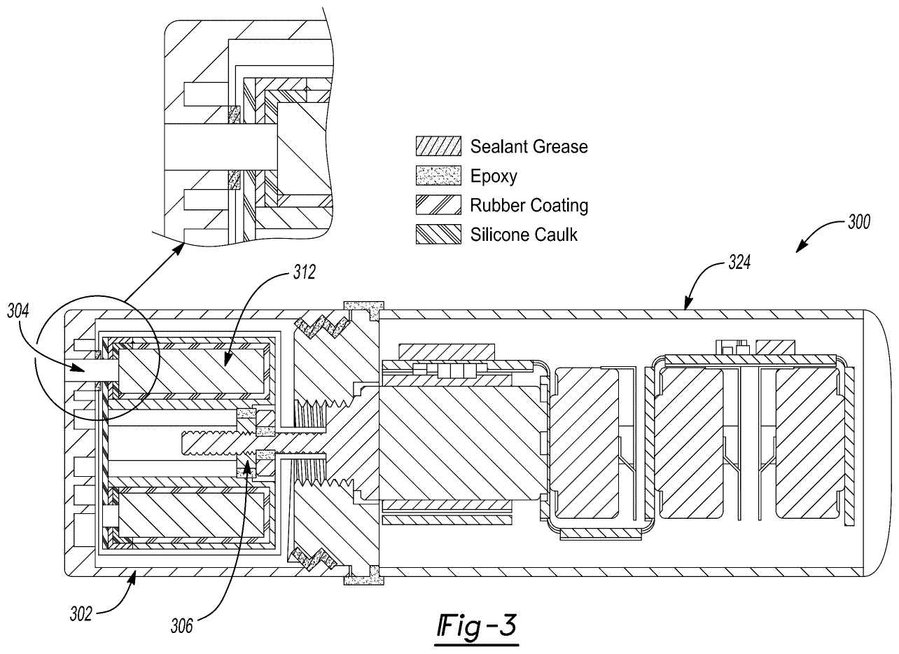

FIG. 2 is an exploded perspective view of another example of an ingestible sampling device 300; whereas, FIG. 3 is a cross-sectional view of the ingestible sampling device of FIG. 2. As in the prior example, the sampling device 300 includes a cap 302 and a housing element 324 that connects thereto. The cap 302 includes a first end or surface 303 having a sampling port 304 defined therewithin.

With continued reference to FIG. 2, the sampling device 300 further includes a front cartridge platform 306 that is disposed adjacent the first surface 303 of the front cap 302 as illustrated in FIG. 3. The front cartridge platform 306 includes a plurality of sampling inlets 308. The sampling inlets 308 of the front cartridge platform 306 and the sampling port 304 of the cap 302 are configured to create channels for the gastric-intestinal fluid samples to flow into one or more of the sample collection chambers or cartridges 312.

The foam cartridges 312 are configured to enhance capillary forces and/or aid filtration of particles within the gastric-intestinal fluid. For example, the foam cartridges 312 may each have a porosity ranging from greater than or equal to about 30% to less than or equal to about 90%, and in certain aspects, optionally greater than or equal to about 30% to less than or equal to about 70%. The pores may have an average diameter ranging from greater than or equal to about 50 nm to less than or equal to about 500 μm. The porosity of the foam cartridges 312 and the diameters of the pores can be varied for difference uses and environments. For example, in certain instances, each foam cartridge 312 may have regional porosity, such that a first portion of the foam cartridge 312 has a first porosity and a first average pore diameter, while a second portion of the foam cartridge 312 has a second porosity and a second average pore diameter. Likewise, each foam cartridge 312 forming the plurality may have a different porosity and/or average pore diameter. In certain aspects, the foam cartridges 312 may also be pretreated to enhance chemical stability or selectivity during the sampling period.

In this fashion, using fluid agitation principals, the transport and storage of the gastric-intestinal fluid samples from a first end 311 to a second end 313 of each foam cartridge 312 may facilitate the creation of a time-correlated profile of the sampled fluid within the foam cartridge 312. For example, gastric-intestinal fluid samples collected first may be stored towards the second end 313 of each foam cartridge 312, while subsequently collected gastric-intestinal fluid samples may be stored towards the first end 311 of each foam cartridge 312. Following expulsion and initial analyses (such as, the creation of a time-correlated profile), the gastric-intestinal fluid samples may be extracted from the foam cartridges 312 using, for example only, a direct withdrawal method, such as a by using a syringe, a centrifugation collection method, and/or a solvent extraction method.

FIG. 4 depicts example circuit components comprising the ingestible sampling device. The circuit components include a stepper motor 41, a motor driver 42, an accelerometer 43 (or another motion sensor), and a microcontroller 44. The ingestible sampling device may further include a wireless communication circuit 45 as well as one or more batteries 46 and power regulation circuits. It is to be understood that only the relevant components of the device are discussed in relation to FIG. 4, but that other circuit components may be needed to control and manage the overall operation of the ingestible device.

In an example embodiment, microcontroller (e.g., CC2650, Texas Instruments, Dallas, TX) is chosen for its small form factor (4×4×1 mm3), number of programmable pins available for system control, and ultra-low power consumption in both the deep sleep state and active state of operation. It may also contain an integrated wireless transceiver that helps simplify the design of the BLE wireless communication circuit and reduce the number of discrete components required. The stepper motor (e.g., FDM0620, Faulhaber, Schönaich, Germany) has an overall size of 06×9.7 mm3 and maximum torque of 0.25 mNm with an mA driving current. The motor has an integrated threaded shaft, allowing easy connection for rotation transmission. The motor driver chip (e.g., LV8044LP, ON Semiconductor, Phoenix, AZ) enables control of the stepper motor in rotation increments as small as 8° while delivering the maximum torque; it also has an ultra-low standby current of 1.0 μA to minimize power consumption when the motor is not in operation. A 3-axis accelerometer (LIS2DH12, STMicroelectronics) is used to sense the XYZ acceleration of the device while it moves along the GI track for location tracking; it provides 3-axis sensing capability with ultra-low-power operation and variable data rate of 1 Hz to 5.3 kHz. The motor driver and accelerometer both interact with the MCU through a standard I2C communication bus. For wireless communication with the external unit, a chip antenna (AH316M245001, Taiyo Yuden, Tokyo, Japan) is used with the MCU wireless transceiver for its small footprint (3.2×1.6 mm2).

Software for the ingestible sampling device is designed to provide three main functions, including motor control for fluid sampling, sensor polling to obtain acceleration data and wireless communication for data transfer. In the example embodiment, motor control is realized using a combination of I2C communication and GPIO control of the LV8044LP motor driver. I2C is also used to communicate with the accelerometer to collect 3-axis acceleration data, which is then saved in the non-volatile flash memory of the MCU. BLE communication is set up such that the ingestible device only advertises and attempts to connect to the external unit after the accelerometer measurements and GI fluid sampling operations have been completed. Once a BLE connection is established, the acceleration data is automatically uploaded to the external unit. All three functions are performed at pre-determined time periods, permitting the ingestible device to remain in the low power state otherwise and conserve battery life.

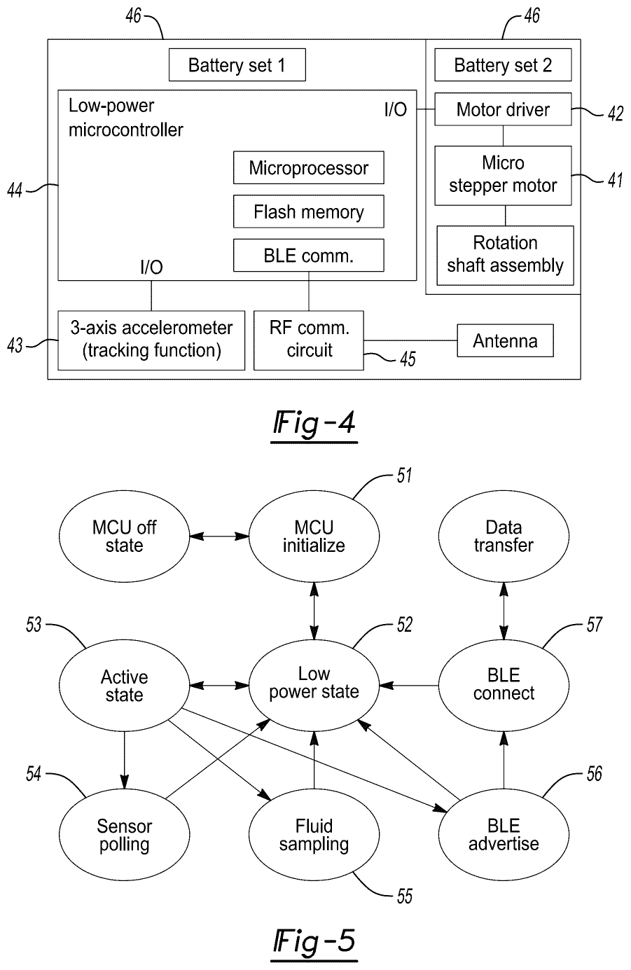

With reference to FIG. 5, an operation flow for the ingestible sampling device is described. Immediately after the MCU is powered on, the BLE communication module and timers controlling different functionalities are initialized at 51, and then the device enters the low power state at 52. When a timer elapses, the MCU enters the active state 53 and performs one of the three main functions. For sensor polling 54, acceleration samples are collected and saved to flash memory; for fluid sampling 55, the motor driver chip is enabled, and the motor is rotated; for BLE advertising 56, the MCU sends advertising packets to attempt to connect with the external unit. If a connection is not established within 60 sec, the MCU returns to the low power state and will attempt to reconnect after a waiting period of 60 sec. When a successful connection to the external unit is established, the MCU enters the connection state 57 and transfers the saved acceleration data. When the external unit is disconnected, the MCU returns to the low power state. The software is set up such that the MCU stays in the low power state for majority of the deployment time to extend battery life.

While exemplary embodiments of sampling device have described above with specific components having specific values and arranged in a specific configuration, it will be appreciated that these devices may be constructed with many different configurations, components, and/or values as necessary or desired for a particular application. The above configurations, components and values are presented only to describe suitable embodiments that have proven effective and should be viewed as illustrating, rather than limiting, the present invention.

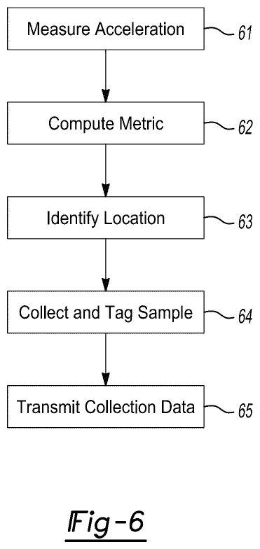

FIG. 6 depicts a method for tracking an ingestible device in a gastrointestinal tract of a subject. As it traverses through the gastrointestinal tract, acceleration of the ingestible device is measured at 61 by a motion sensor disposed in the ingestible device. In one example, the motion sensor is an accelerometer although other types of motion sensors are contemplated by this disclosure. In the example embodiment, the ingestible device is the sampling device 100 described above. While reference is made throughout this disclosure to this particular sampling device, it is readily understood that the tracking technique described herein is applicable to any type of ingestible device that is able to measure and process its acceleration as it moves through the gastrointestinal tract.

Next, a metric is computed at 62 from the acceleration measurements obtained over a given period of time and a location of the ingestible device in the gastrointestinal tract is identified at 63 based in part on the metric. As shown in the test results discussed below, metrics derived from acceleration will vary depending on the location of ingestible device in the gastrointestinal tract. In one example, a peak value of acceleration will differ amongst the different regions of the gastrointestinal tract. In this case, the peak value of acceleration is higher in the small intestine than in the stomach or in the large intestine. In another example, an acceleration spread is derived from the acceleration measurements, where the acceleration spread is the range of the acceleration measurements during the given period of time. Likewise, the acceleration spread is higher in the small intestine than in the stomach or in the large intestine.

For illustration purposes, an example technique will be described for distinguishing between the small intestine, large intestine and stomach of the gastrointestinal tract. This technique assumes a determination has been made as to when the ingestible device entered the stomach. Such determination may be made, for example using elapsed time from when the ingestible device entered the gastrointestinal tract or some other method. As the ingestible device traverses through the stomach, a metric derived from the acceleration measurements (e.g., range or peak value) is continually computed and compared to a first threshold, where the first threshold indicates transition of the ingestible device from the stomach to the small intestine. Once the metric exceeds the threshold, the location of the ingestible device is identified as being in the small intestine. The location of the ingestible device may be determined solely from the metric or from the metric in combination with other indicators, such as elapsed time from when the ingestible device entered the gastrointestinal tract.

As the ingestible device traverses through the small intestine, the metric derived from the acceleration measurements continues to be computed. The metric is now compared to a second threshold, where the second threshold indicates transition of the ingestible device from the small intestine to the large intestine. In this case, once the metric is below the second threshold, the location of the ingestible device is identified as being in the large intestine. Again, the location of the ingestible device may be determined solely from the metric or from the metric in combination with other indicators, such as elapsed time from when the ingestible device entered the gastrointestinal tract.

With continued reference to FIG. 6, the ingestible device may operate to collect sample from the gastrointestinal tract as indicated at 64. In this case, the collected sample can be tagged with the location in the gastrointestinal tract at which the sample was collected. After the ingestible device has passed through the subject, the collected sample as well as metadata related to the collected sample (including its collection location) can be obtained from the ingestible device. Elapsed time from when the ingestible device entered the gastrointestinal tract is another example of metadata that can be collected by the ingestible device. Additionally or alternatively, the metadata can be communicated wirelessly from the ingestible device at 65 as it passes through the gastrointestinal tract to another device remotely located outside of the gastrointestinal tract.

Instead of collecting samples from the gastrointestinal tract, the ingestible device may be configured to release a substance from the ingestible device while at the particular location in the gastrointestinal tract. Similarly, the release location of the substance may be recorded by the ingestible device. After the ingestible device has passed through the subject, the release location along with other metadata can be obtained from the ingestible device. Additionally or alternatively, the release location and other metadata can be communicated wirelessly from the ingestible device as it passes through the gastrointestinal tract to another device remotely located outside of the gastrointestinal tract.

The ingestible sampling device 100 was extensively tested in vivo using mongrel hounds. These tests were performed to characterize the transit time of the ingestible device through the GI tract, evaluate effectiveness of the sealing approaches, and verify sampling and location tracking functions of the ingestible device. The ingestible device was then orally administered. Radiographs were regularly taken to monitor the approximate location of the ingestible device in the GI tract during the tests. After the ingestible device was expelled by the hound and retrieved, the device was cleaned and the device cap was detached by unthreading from the lock ring. The cartridge assembly was removed and disassembled to allow the sample cartridges to be collected. The fluid sample collected in each cartridge was extracted by centrifuge. Volume measurement and LC-MS were then carried out on the samples to analyze the concentrations of the drug and bile salt. The acceleration data was also wirelessly read out through a wireless communication link (i.e., BLE) to a computer for postprocessing.

During eight in vivo tests, transit times of the ingestible device were recorded. The gastric emptying time (GET) values were estimated to be around 5-20 hours in some dogs and 20-48 hours in other dogs. It is worth noting that it is challenging to accurately calculate GET based on 2-3 radiographs and the long GET can be due to the time gap between the radiography performed before the end of business hours on the first day of deployment and the next after the start of the second day. The total GI transit time for the hounds was 20-70 hours. The distribution of the transit time provided guidance for programming the ingestible device and tailoring its operation lifetime necessary to fit the deployment needs.

TABLE 1
Ingestible device transit results in mongrel hounds.

| | Gastric | |
|---|---|---|
| Experiment | Emptying | Total Transit |
| Number | Time (h) | Time (h) |
| 1 | 25-48 | 70 |
| 2 | 8-25 | 25-48 |
| 3 | 0-12 | 0-12* |
| 4 | 19-37 | 37-60 |
| 5 | 5-24 | 5-24* |
| 6 | 5-24 | 27 |
| 7 | 5-24 | 5-24* |
| 8 | 5-24 | 24-48 |

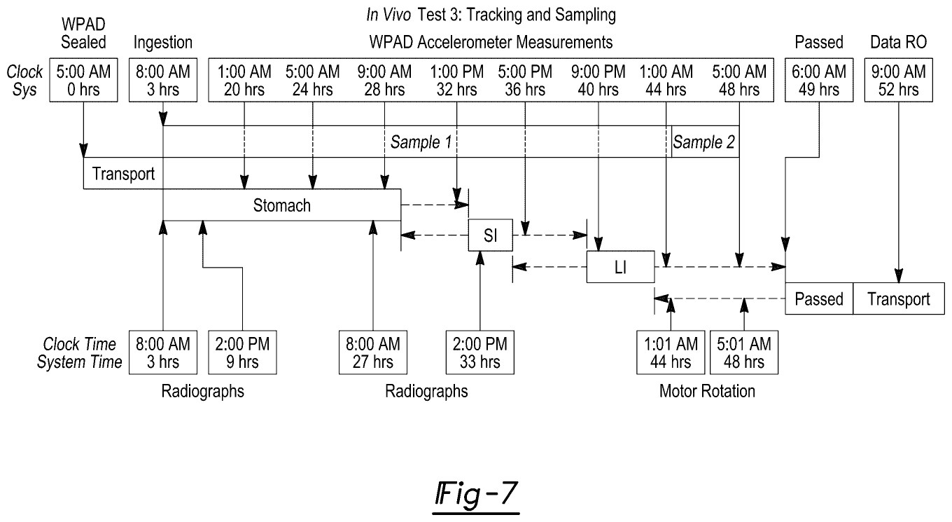

A series of in vivo tests of the ingestible device using hounds for both sampling and the tracking functions were performed. For these tests, the ingestible device was programmed to collect acceleration data and take fluid samples by rotating the internal cartridge assembly. The results from one of the tests (In Vivo Test 3) are shown in FIG. 7. In this test, the fully assembled and sealed ingestible device was administered with 500 mg Pentasa (mesalamine) to a two-year old mongrel hound named Molly weighing ≈60 pounds. An initial delay of 20 hr was used before taking any accelerometer readings to account for time for sealing, transit, ingestion, and stomach residence. After the initial delay, eight acceleration readings were taken in 30 measurement bursts every 4 hr. The sampling port on the device cap was initially aligned to the first foam cartridge, allowing sample in the stomach to be collected. At Hour 44, the cartridge assembly was rotated to align the second foam cartridge to the sampling port, stayed in the position for 4 hours targeting the sample in the colon and then moved to Position C at Hour 48. Radiographs were taken to observe the ingestible device location as it passed through the GI tract. The ingestible device was passed overnight with a transit time between 43 and 49 hours. The ingestible device was interrogated and the acceleration data was retrieved. The sampling foam cartridges were removed from the ingestible device to extract the collected samples for analysis.

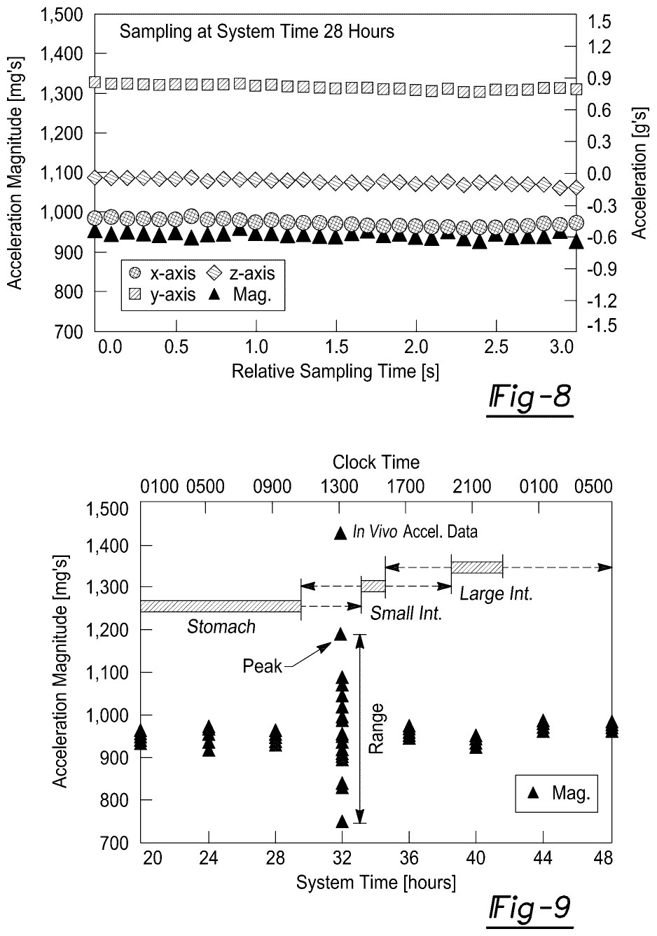

With reference to FIGS. 8 and 9, recorded acceleration data demonstrates distinct motion patterns in different segments of the GI tract. When the ingestible device was in the stomach, the range of variation in the acceleration magnitude was modest; however, when the ingestible device passed from the stomach to the small intestine, a distinct increase in the magnitude of the acceleration could be observed, which then diminished when the ingestible device entered the large intestine. Typical results of the spread of the acceleration magnitude and peak of the acceleration from the In Vivo Test 3 are summarized in Table 2 below.

TABLE 2
location and acceleration values of in vivo Test 3

| | | Accel. | Accel. |
|---|---|---|---|
| Time | | Spread | Peak |
| [hr] | Region | (milli-g) | (milli-g) |
| 20 | Stomach | ≈30 | 957 |
| 24 | Stomach | ≈50 | 972 |
| 28 | Stomach | ≈30 | 960 |
| 32 | Small | ≈600 | 1442 |
| | Intestine | | |
| 36 | Large | ≈30 | 973 |
| | Intestine | | |
| 40 | Large | ≈20 | 949 |
| | Intestine | | |
| 44 | Large | ≈20 | 983 |
| | Intestine | | |
| 8 | Large | ≈20 | 983 |
| | Intestine | | |

Different regions of the GI tract demonstrated different acceleration magnitude patterns. It could be observed that the peak acceleration magnitude and the spread in the acceleration (30-50 milli-g) in stomach were relatively low, matching expected modest motion pattern in stomach. However, when the ingestible device moved into the small intestine, the peak acceleration magnitude and the spread (600 milli-g) increased significantly, which matched the characteristic high rate of transit in the small intestine and could be used as motility signatures to identify the GI segment location of the ingestible device. As the ingestible device moved into the large intestine, the peak acceleration magnitude and the spread in acceleration magnitude (20-30 milli-g), were once again diminished. These acceleration recordings were found to be typical and repeatable among all in vivo tests of the ingestible device, indicating a characteristic pattern of the acceleration in different segments of the GI tract that can be used to identify the GI tract segment.

Measurement of drug release and bile salts from samples collected during in vivo sampling tests. In order to test the sampling function of the ingestible device, the ingestible device was also administered with modified release oral drug product Pentasa that contains 500 mg mesalamine. The expelled ingestible device was retrieved to collect the samples to measure drug concentration. The rationale to use Pentasa is that this drug is designed to have very low drug release in the stomach, but high rate of drug release in small intestine and in the colon.

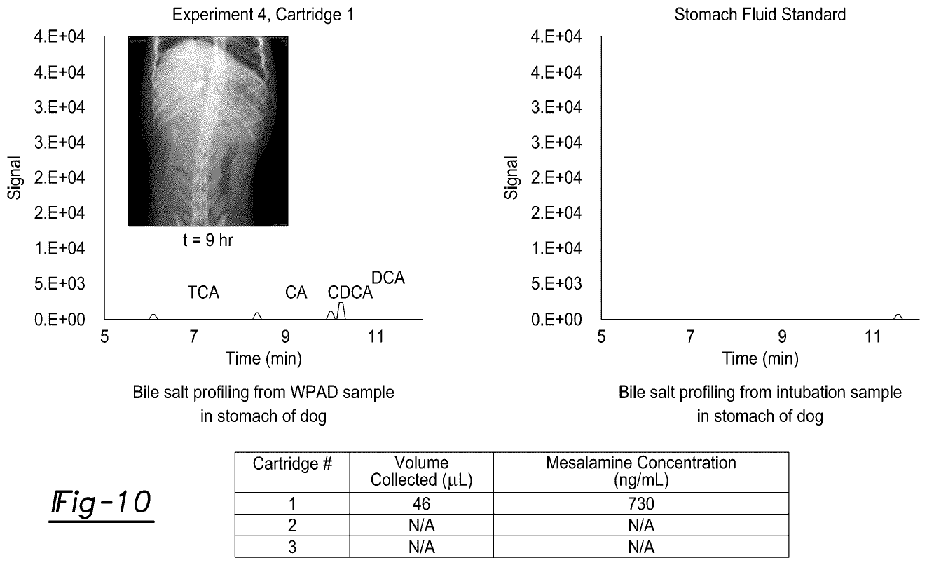

The collected fluids during the in vivo tests were extracted by centrifuge and then assayed for mesalamine concentration and bile acid content. FIG. 10 shows results from Cartridge 1 in Experiment 4 targeting sample collection in stomach. The concentration of mesalamine in the chamber is 730 ng/mL. This sample had a low bile salt profile that appears to be from stomach. These bile acids in combination with the radiographs could indicate that the samples are from the stomach.

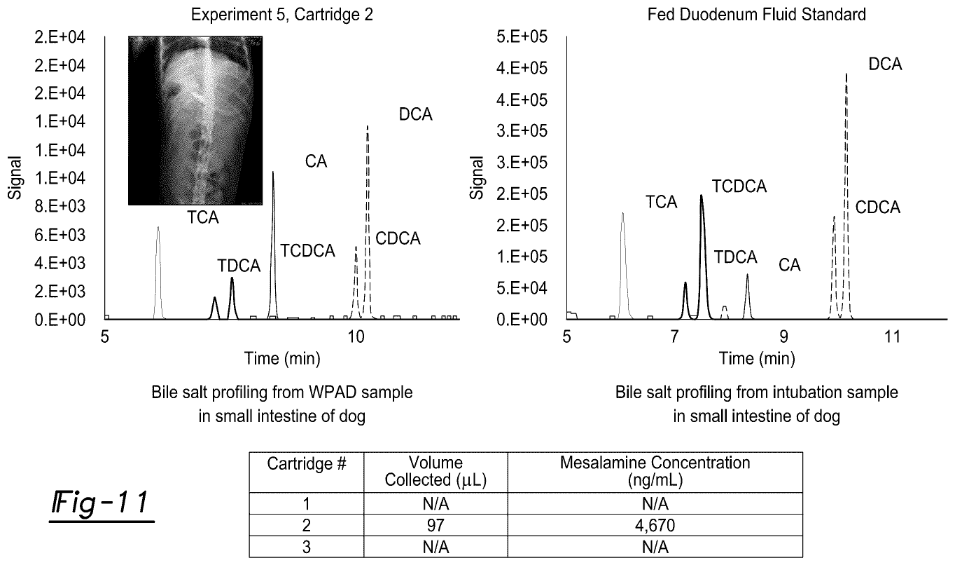

FIG. 11 shows results from Cartridge 2 in Experiment 5 targeting sample collection in small intestine. The hound used in this experiment was the same as in Experiment 3, and there was much higher mesalamine concentration in both experiments at levels expected for in vivo dissolution. The bile acid profile of this sample matches the fed state fluid from the duodenum. This indicates that the sample came from the duodenum shortly after the hound had eaten. The radiographs also indicate that the sample was taken when the ingestible device was in the duodenum.

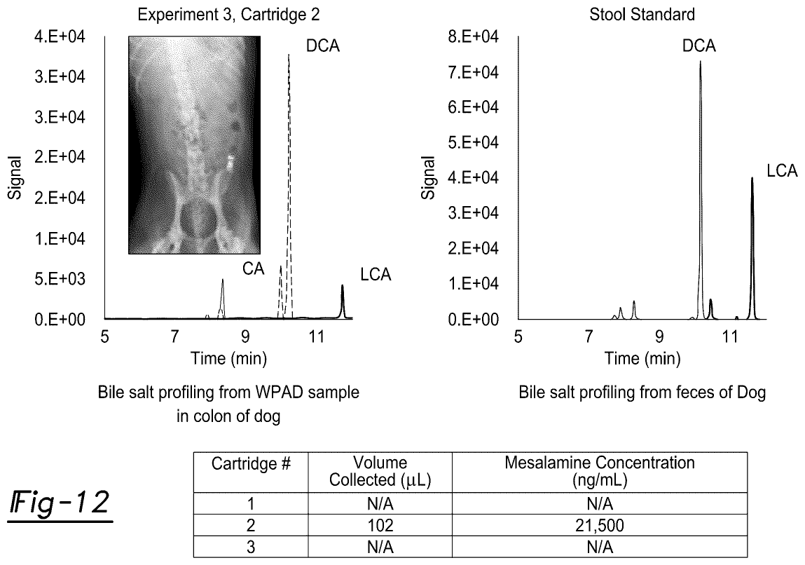

FIG. 12 shows results from Cartridge 2 in Experiment 3 targeting sample collection in large intestine. The mesalamine concentration matches the expected level in dogs and therefore the value is plausible. The bile acid profiles also help confirm the origin of the samples. The presence of LCA in the sample indicates it is likely from the colon, which is also confirmed by the radiographs.

TABLE 3
comparison of ingestible GI tract sampling device

| | | Fluid | | | | |
|---|---|---|---|---|---|---|
| | Sampling | Sampling | Commu- | Location | Capsule | Animal |
| Device | Mechanism | Capability | nication | Tracking | Size | Species |
| WPAD | Capillary | 3 × 15 | RF (BLE) | Embedded | Ø14 × | Canine/ |
| (this | action | μL | | micro- | 42 | Hound |
| work) | | (triggered | | accelermeter | mm² | |
| | | by stepper | | | | |
| | | motor) | | | | |
| Cui | Vacuum | 1 × 262 | RF | Embedded | Ø11 × | Porcine |
| capsule | sorption | μL | | magnet | 30 | |
| [22] | | (triggered | | | mm² | |
| | | by MEMS | | | | |
| | | calorific | | | | |
| | | element) | | | | |
| Nejad | Osmotic | 1 × 120 | No | Embedded | Ø9 × | Porcine |
| pill | pumping | μL | | magnet | 24 | & Macaque |
| [24] | | (triggered | | | mm² | |
| | | by disso- | | | | |
| | | lution of | | | | |
| | | enteric | | | | |
| | | capsule) | | | | |

The ingestible device reported in this disclosure is compared in Table 3 above to other devices with in vivo results for gastrointestinal sampling. The ingestible device was able to collect up to three GI tract fluid samples stored in individual foam cartridges housed inside the ingestible device. Selection between foam cartridges was achieved by incorporating a stepper motor to rotate the capsules in order to align them with the sampling port of the device cap. Inherent to foam, sample collection is accomplished by capillary action. The overall size of the ingestible device was similar to other devices described in Table 3 with a diameter of 14 mm and length of 42 mm. Housing components were 3D printed using a biocompatible resin and the device was sealed using silicone grease.

The housing materials and sealing method was demonstrated to be effective in preventing leakage and enable sampling for the ingestible device. Lastly, the ingestible device was capable of BLE communication with an extra-corporeal unit to send relative data as well as enable wireless triggering of the motor for sampling by the extra-corporeal unit.

The acceleration data successfully collected from the GI tract deployment represents the first effort in exploring the use of the acceleration pattern for location tracking inside the GI tract. The results suggest that it is feasible to identify motion patterns in different segments of the GI tract using an accelerometer and, therefore, identify the segment location of the ingestible device during its GI tract transit. The acceleration data demonstrates the motility within different regions of the GI tract and can be used to permit the ingestible device to autonomously identify which region it is in, permitting sampling in different regions of the GI tract without requiring external control or radiograph tracking. It is also notable that the recorded acceleration of the ingestible device is not affected by the activity of the hound, diminishing the need for an external reference unit that is worn by the hound to monitor its body acceleration. This is because the GI tract presents a highly damped environment that protects the ingestible device from external shock, and the further encapsulation of the accelerometer within the ingestible device provides additional isolation.

By virtue of the disclosure, it was observable that the spread (or range) and the peak acceleration magnitude of the ingestible device both increased significantly in the small intestine, reflecting the unique characteristic high rate of transit in the small intestine. To confirm if this indeed reflect the transit of small intestine, PillCam was used to visualize the movement of Pentasa in the small intestine vs. stomach. Indeed, the Pentasa granules transited into the small intestine in a much faster back and forth motion than that in the stomach. The increased acceleration magnitude recorded by ingestible device may reflect these movement.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for tracking an ingestible device in a gastrointestinal tract of a subject, comprising:
measuring, by an accelerometer disposed in the ingestible device, acceleration of the ingestible device as it traverses through the gastrointestinal tract;
computing, by a processor, a metric from the acceleration measurements obtained by the accelerometer over a given period of time; and
identifying, by the processor, a location of the ingestible device in the gastrointestinal tract based in part on the metric.

2. The method of claim 1 wherein computing a metric further comprises determining a range of the acceleration measurements during the given period of time.

3. The method of claim 2 further comprises identifying the location of the ingestible device in the gastrointestinal tract as small intestine in response to the range of acceleration measurements exceeding a threshold.

4. The method of claim 3 further comprises identifying the location of the ingestible device in the gastrointestinal tract as large intestine in response to the range of acceleration measurements being less than a second threshold.

5. The method of claim 1 wherein computing a metric further comprises determining a peak value of the acceleration measurements during the given period of time.

6. The method of claim 5 further comprises identifying the location of the ingestible device in the gastrointestinal tract as small intestine in response to the peak value of acceleration measurements exceeding a threshold.

7. The method of claim 6 further comprises identifying the location of the ingestible device in the gastrointestinal tract as large intestine in response to the peak value of acceleration measurements being less than a second threshold.

8. The method of claim 1 further comprises collecting a sample from the gastrointestinal tract using the ingestible device and tagging the sample with the location in the gastrointestinal tract at which the sample was collected.

9. The method of claim 1 further comprises determining when the ingestible device passes through a particular location in the gastrointestinal tract and releasing a substance from the ingestible device while at the particular location.

10. The method of claim 1 further comprises wirelessly communicating the location in the gastrointestinal tract to another device outside of the gastrointestinal tract.

11. The method of claim 1 further comprises
wirelessly communicating the acceleration measurements to another device outside of the gastrointestinal tract;
computing, by the processor, the metric from the acceleration measurements obtained by the accelerometer over a given period of time; and
identifying, by the processor, the location in the gastrointestinal tract based in part on the metric, where the processor resides in the another device.

12. The method of claim 1 further comprises
monitoring elapsed time from when the ingestible device entered the gastrointestinal tract; and
tagging the location in the gastrointestinal tract with an elapsed time at which the ingestible device enters the location in the gastrointestinal tract.

13. A method for tracking an ingestible device in a gastrointestinal tract of a subject, comprising:
measuring, by an accelerometer disposed in the ingestible device, acceleration of the ingestible device as it traverses through the gastrointestinal tract;
wirelessly communicating acceleration measurements from the ingestible device to another device outside of the gastrointestinal tract;
computing, by a processor of the another device, a metric from the acceleration measurements; and
identifying, by the processor, the location of the ingestible device in the gastrointestinal tract based in part on the metric.

14. An ingestible device for traversing a gastrointestinal tract, comprising:
a housing configured to be ingested by a subject;
an accelerometer disposed inside the housing and operable to measure acceleration of the ingestible device as it traverses through the gastrointestinal tract of the subject; and
a microcontroller residing in the housing and interfaced with the accelerometer, wherein the microcontroller is configured to computer a metric from the acceleration measurements obtained by the accelerometer over a period of time and identify a location of the ingestible device in the gastrointestinal tract based on the metric.

15. The ingestible device of claim 14 wherein the microcontroller determines a range of the acceleration measurements during the given period of time; identifies the location of the ingestible device in the gastrointestinal tract as small intestine in response to the range of acceleration measurements exceeding a threshold; and identifies; and the location of the ingestible device in the gastrointestinal tract as large intestine in response to the range of acceleration measurements being less than a second threshold.

16. The ingestible device of claim 14 wherein the microcontroller computes determines a peak value of the acceleration measurements during the given period of time; identifies the location of the ingestible device in the gastrointestinal tract as small intestine in response to the peak value of acceleration measurements exceeding a threshold; and identifies the location of the ingestible device in the gastrointestinal tract as large intestine in response to the peak value of acceleration measurements being less than a second threshold.

17. The ingestible device of claim 14 further comprises a wireless transceiver interface with the microcontroller and wirelessly communicates the location in the gastrointestinal tract to another device located outside of the gastrointestinal tract.

18. The ingestible device of claim 14 further comprises
a cap connected to the housing and comprising a surface defining a sampling port;
one or more sample collection chambers within the housing and configured to collect the gastric-intestinal samples;
a rotatable shaft disposed within the housing and is configured to rotate one of the cap or the one or more sample collection chambers; and
a motor connected to the rotatable shaft within the housing and configured to axially rotate the rotatable shaft; wherein the microcontroller is configured to control the motor and rotatable shaft so as to selectively align the sampling port of the cap with at least one of the sample collection chambers, thereby exposing the at least one sample collection chamber to the gastric-intestinal tract for collection of one or more gastrointestinal fluid samples.

19. The ingestible device of claim 18 wherein the microcontroller operates to collect samples from the gastrointestinal tract and tag the samples with the location in the gastrointestinal tract at which the sample was collected.

20. The ingestible device of claim 14 wherein the microcontroller monitors elapsed time from when the ingestible device entered the gastrointestinal tract; and tags the location in the gastrointestinal tract with an elapsed time at which the ingestible device enters the location in the gastrointestinal tract.
